(19) United States
(12) Patent Application Publication
(10) Pub. No.: US 2025/0339293 A1
(43) Pub. Date: Nov. 6, 2025

(54) PROSTHETIC FOOT COVER ASSEMBLY

(71) Applicant: Össur Iceland ehf, Reykjavik (IS)

(72) Inventors: Arinbjörn Viggo Clausen, Reykjavik (IS); Felix Starker, Reykjavik (IS)

(21) Appl. No.: 18/871,740

(22) PCT Filed: Jun. 2, 2023
(86) PCT No.: PCT/IB2023/055714

§ 371 Date: Dec. 4, 2024

Related U.S. Application Data
(60) Provisional application No. 63/365,894, filed on Jun. 6, 2022.

(51) Int. Cl. A61F 2/66; A61F 2/50

(52) U.S. Cl. CPC A61F 2/66; A61F 2002/5081

(57) ABSTRACT

A water-tight foot cover assembly is described. In one example, the water-tight foot cover includes a foot cover, and a seal. The foot cover can have a pattern on a bottom surface to increase friction between the water-tight foot cover and a walking surface. The seal can include one or more one-way valves and provide a water-tight seal so that water, dirt, or other debris cannot enter an inner cavity of the foot cover. The water-tight foot cover assembly can include an inner shell that provides structural support to the foot cover. The foot cover can be pressurized or vacuumized in order to change structural properties of the foot cover.

### INCORPORATION BY REFERENCE TO ANY PRIORITY APPLICATIONS

The present application is a National Phase Application of PCT/IB2023/055714 filed Jun. 2, 2023, titled PROSTHETIC FOOT COVER ASSEMBLY, which claims priority to U.S. Provisional Patent Application No. 63/365,894, filed Jun. 6, 2022, which is incorporated herein by reference. Any and all applications for which a foreign or domestic priority claim is identified in the Application Data Sheet as filed with the present application are hereby incorporated by reference under 37 CFR 1.57.

### BACKGROUND

### Field

The present application relates to foot covers in general, and more particularly, aspects of the present application relate to water-tight foot covers and foot cover assemblies configured to provide increased friction between the foot cover and a walking surface.

### Description

Conventional prosthetic foot covers are designed to cover prosthetic feet and are typically manufactured out of one piece of material and include an opening for insertion of a prosthetic foot. However, during use, water, dirt, or other debris may enter the interior of the foot cover via the opening. The water, dirt, or other debris may cause accelerated wear of portions of the prosthetic foot. For example, dirt may get lodged between two parts of the prosthetic foot, and the dirt may cause additional friction. Furthermore, conventional foot covers do not provide grip or may provide limited grip when walking on uneven or wet surface, such as dirt or mud, and are designed to be worn with a shoe protecting the foot cover. Therefore, the materials used for making the foot cover can wear down or crack when the foot cover is used on rough, uneven, or wet surfaces without a shoe. However, in rural areas, shoes may not always be accessible.

Accordingly, there is a need for an improved foot cover and/or foot cover assembly that can provide a water-tight seal to keep liquid, dirt or other debris out of the foot cover and can provide additional friction between the foot cover and a walking surface to facilitate ambulation.

### SUMMARY

In accordance with one aspect of the disclosure, a water-tight and debris tight foot cover and/or foot cover assembly that is made out of resilient material is provided that has less wear or is more durable when the foot cover is used on rough, uneven, or wet surfaces compared to existing foot cover products.

In accordance with another aspect of the disclosure, a water-tight and debris tight foot cover and foot cover assembly/or is provided that is made out of resilient material, and that can be used without a shoe or sandal to walk on rough, uneven, or wet surfaces, the foot cover inhibiting wear and tear and cracking when used on such surfaces and having sole features that facilitate friction during ambulation on such surfaces.

In some configurations, a prosthetic foot cover assembly can include: a foot cover including a bottom surface, a top opening and an inner cavity, wherein the inner cavity is configured to receive a prosthetic foot through the top opening; and a seal removably coupleable to the foot cover proximate the top opening, wherein the bottom surface includes a pattern of indentations, and the top opening includes at least one protrusion, and wherein the seal has substantially a same shape as the top opening such that the seal and the at least one protrusion inhibit liquids, solids and/or gases from entering the inner cavity.

In some configurations, the pattern of indentations can extend along an entirety of the bottom surface.

In some configurations, the pattern of indentations can be configured to increase friction between the bottom surface and a walking surface.

In some configurations, the at least one protrusion can be on an inner surface of the cover and the at least one protrusion can define a channel that couples to at least a portion of the seal.

In some configurations, a leg cosmesis can be coupled to the foot cover.

In some configurations, the leg cosmesis can be press fit to the foot cover.

In some configurations, the foot cover can include a recess in an outer surface of the foot cover at the top opening, and the recess can be configured to receive a protrusion on an inner surface of the leg cosmesis in order couple the leg cosmesis to the foot cover.

In some configurations, adhesive can be applied to the foot cover and/or the leg cosmesis to secure the protrusion of the leg cosmesis to the recess of the foot cover.

In some configurations, the channel can be a first channel and the at least one protrusion can define a second channel that couples to at least a portion of a retainer.

In some configurations, the second channel can couple to a first portion of the retainer, and the retainer can be positioned so a second portion of the retainer extends above the top opening of the cover.

In some configurations, the second portion of the retainer can be configured to removably couple a leg cosmesis to the foot cover.

In some configurations, the leg cosmesis can be shaped to resemble a human leg.

In some configurations, the leg cosmesis can extend the top opening of the foot cover to a position above a knee of a human leg.

In some configurations, the leg cosmesis can extend from the top opening of the cover to a position below a knee of a human leg.

In some configurations, the foot cover can include one or more of rubber, silicone, nitrile, polyvinyl chloride, or neoprene.

In some configurations, the prosthetic foot cover assembly can include an inner shell configured to receive the prosthetic foot, wherein the inner shell is disposed in the inner cavity.

In some configurations, the inner shell can include one or more of foam and a plastic material.

In some configurations, the inner shell can include a recess positioned at a heel end of the inner shell, and the recess can be configured to receive an insert.

In some configurations, the inner shell can include a first material and the insert can include a second material.

In some configurations, the second material can be stiffer than the first material.

In some configurations, the inner shell can include an open cell structure.

In some configurations, the inner shell can include a plurality of interconnected cells.

In some configurations, the seal can include a seal opening configured to wrap around an adaptor of the prosthetic foot.

In some configurations, the adaptor of the prosthetic foot can be configured to coupled a prosthetic leg to the prosthetic foot.

In some configurations, the seal opening can be configured to provide an airtight seal with the adaptor.

In some configurations, the adaptor can include a recess configured to receive a gasket.

In some configurations, the seal can include a first one-way valve configured to let air into the inner cavity.

In some configurations, the prosthetic foot cover assembly can include a tube coupleable to the first one-way valve and configured to extend from the first one-way valve into the inner cavity.

In some configurations, the seal can include a second one-way valve configured to let air out of the inner cavity.

In some configurations, the first one-way valve and the second one-way valves can be configured to pressurize the inner cavity.

In some configurations, the prosthetic foot cover can include one or more portions of inelastic material coupled to one or more portions of an inner surface of the inner cavity.

In some configurations, the first one-way valve and the second one-way valves can be configured to maintain the inner cavity under a vacuum.

In some configurations, the prosthetic foot cover assembly can include a vacuum bag coupled to one or both of the first one-way valve and the second one-way valve.

In some configurations, a toe end of the foot cover can include toes shaped to resemble a shape of human toes.

In some configurations, the toes can each include a nail including a polished surface.

In some configurations, the nail can be configured to be coupled with an artificial nail.

In some configurations, the toes can be configured to engage a raised surface of a footwear.

In some configurations, the toe end of the foot cover can include a first toe positioned on a medial side of the toe end and a second toe positioned laterally to the first toe, and the foot cover can include a gap between the first toe and the second toe.

In some configurations, the gap can be configured to receive a toe post of a footwear when the prosthetic foot cover assembly is inserted into the footwear.

In some configurations, the foot cover can include a top surface, and the top surface can include a texture.

In some configurations, the texture can be configured to bond ink or dye to the top surface.

In some configurations, the cover can include a color, and the color can be a color of a user's skin.

In some configurations, the color of the cover can be boiled into the cover.

In some configurations, an inner shell for a prosthetic foot cover assembly can include: a body including a plurality of interconnected cells; and a foot cavity in the body, the foot cavity configured to receive a prosthetic foot, wherein the inner shell is configured to be inserted into an inner cavity of a prosthetic foot cover, and wherein a cell density of the plurality of interconnected cells varies throughout the body so a stiffness of the body varies throughout the body.

In some configurations, each cell of the plurality of interconnected cells can include struts, and the struts can define an opening of each cell.

In some configurations, the stiffness of the body can vary throughout the body to approximate a stiffness a human foot.

In some configurations, the body can include a bottom surface, the bottom surface including a width and a length, and each cell of the plurality of interconnected cells can flex, expand, and/or compress so the width and/or the length can increase when a force is applied to the bottom surface.

In some configurations, a heel portion of the body can include a solid structure.

In some configurations, the heel portion of the body can include a plurality of protrusions.

In some configurations, when the prosthetic foot cover and the inner shell are inserted into footwear, each cell of the plurality of interconnected cells can flex, expand, and/or compress based on a geometry of the footwear.

In some configurations, the cell density of the plurality of interconnected cells can be determined based on a stiffness and/or an anatomy of a foot of a user.

In some configurations, a toe end of the inner shell can be shaped to resemble a shape of human toes.

### BRIEF DESCRIPTION OF THE DRAWINGS

These and other features, aspects, and advantages of the present disclosure are described with reference to the drawings of certain aspects, which are intended to schematically illustrate certain aspects and not to limit the disclosure.

FIG. 1A illustrates a perspective view of an example of a water-tight foot cover with an example prosthetic foot received therein.

FIG. 1B illustrates a bottom view of an example foot cover of FIG. 1A illustrating an example bottom surface.

FIG. 1C illustrates a cross-section of the foot cover of FIG. 1B illustrating an inner cavity of the foot cover.

FIG. 2A illustrates a perspective view of an example of a water-tight seal of a water-tight foot cover assembly.

FIG. 2B illustrates a perspective view of an example of a water-tight seal of a water-tight foot cover assembly with a tube.

FIG. 2C illustrates a cross-section of a water-tight seal of a water-tight foot cover assembly with a tube.

FIG. 3 illustrates a cross-section of an example water-tight foot cover assembly coupled to a prosthetic foot.

FIG. 4A illustrates a perspective view of an example of an inner shell of an example water-tight foot cover assembly.

FIG. 4B illustrates a bottom view of an inner shell of an example water-tight foot cover assembly.

FIG. 4C illustrates a cross-section of an example water-tight foot cover assembly with an inner shell.

FIG. 5A illustrates a perspective view of an example water-tight foot cover assembly.

FIG. 5B illustrates a bottom view of the example water-tight foot cover assembly of FIG. 5A.

FIG. 5C illustrates a side view of the example water-tight foot cover assembly of FIG. 5A.

FIG. 5D illustrates in a detailed view a textured surface of an example foot cover of the water-tight foot cover of FIG. 5A.

FIG. 5E illustrates a cross-section of a foot cover of the water-tight foot cover assembly of FIG. 5A illustrating an inner cavity of the foot cover.

FIG. 6A illustrates a perspective view of an example of a retainer of the foot cover assembly of FIG. 5A.

FIG. 6B illustrates a side view of the retainer of FIG. 6A.

FIG. 7A illustrates a cross-section of the example water-tight foot cover assembly (including an inner shell) and a prosthetic foot coupled to the example water-tight foot cover assembly.

FIG. 7B illustrates a perspective view of an example top portion of an adaptor of the water-tight foot cover assembly of FIG. 7A.

FIG. 8A illustrates a perspective view of an example of a leg cosmesis.

FIG. 8B illustrates a cross-section of the example leg cosmesis of FIG. 8A.

FIGS. 9A-9C illustrate various example leg cosmeses coupled to an example water-tight foot cover.

FIG. 10A illustrates a perspective view of an example of an inner shell of a water-tight foot cover assembly.

FIG. 10B illustrates a cross section of the inner shell of FIG. 10A.

FIG. 11A illustrates a perspective view of an example of an inner shell.

FIG. 11B illustrates a partial bottom view of the inner shell of FIG. 11A.

FIG. 11C illustrates a partial perspective view of an example inner shell.

FIG. 11D illustrates a partial perspective view of an example inner shell.

FIG. 12 illustrates a partial sectional view of an example leg cosmesis coupled to an example water-tight foot cover.

FIG. 13 illustrates a schematic of a wedge of the inner shell of FIG. 11A.

### DETAILED DESCRIPTION

These and other features, aspects, and advantages of the present disclosure are described with reference to the drawings of certain aspects, which are intended to schematically illustrate certain aspects and not to limit the disclosure.

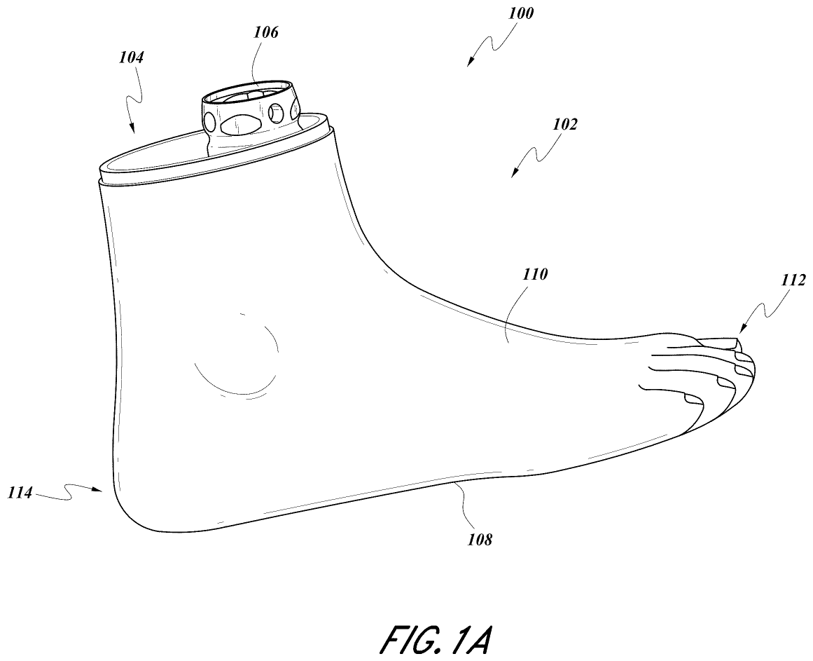

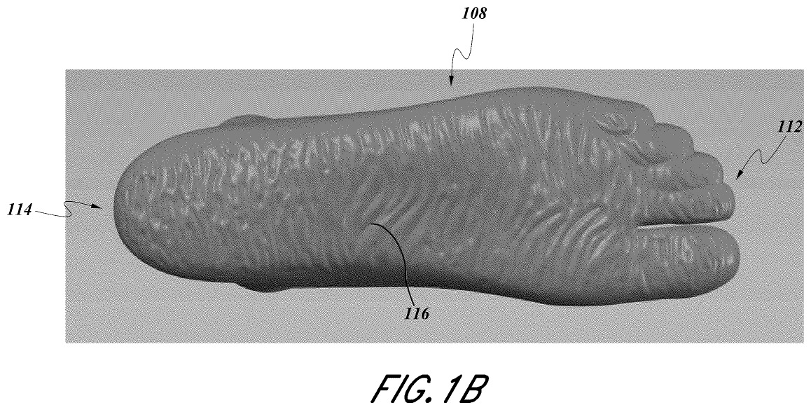

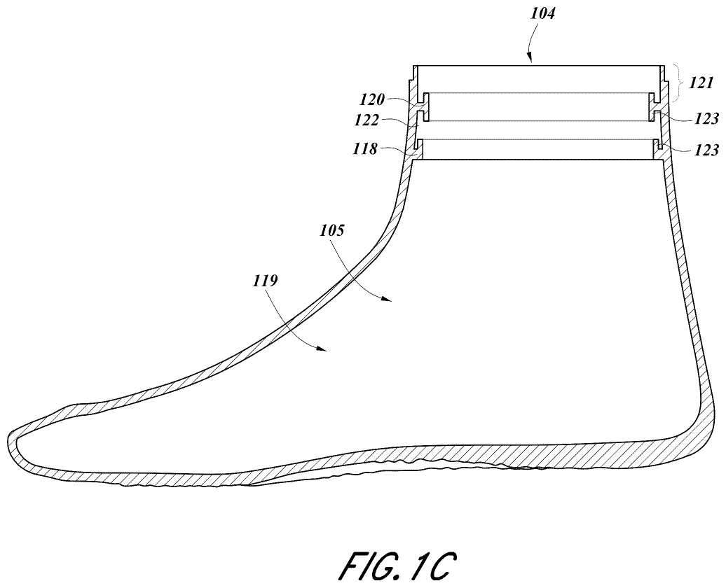

FIGS. 1A-1C illustrate an example of a foot cover assembly 100. As will be described in greater details below, the foot cover assembly can include, in addition to a foot cover, other components such as a seal, an inner shell, etc. The foot cover assembly 100 can include a foot cover 102. As shown in FIG. 1A, the foot cover 102 extends from a toe end 112 to a heel end 114. The foot cover 102 can include an opening 104 at or near an ankle location of a prosthetic foot 106 when the prosthetic foot 106 is inserted into the foot cover 102. The prosthetic foot 106 can be inserted into an inner cavity of the foot cover 102 via the opening 104. The foot cover 102 can be made of rubber, silicone, nitrile, polyvinyl chloride, neoprene, or any other water-resistant rubber replacement material. In some aspects, the foot cover 102 can have a hardness. The hardness of the foot cover 102 and/or other components of the foot cover assembly disclosed herein can be determined with a durometer. The foot cover 102 can have a hardness of about 10A (i.e., 40 00), about 20A (i.e., 50 00), about 30A (i.e., 60 00), about 40A (i.e., 70 00), about 50A (i.e., 80 00, 10D), about 60A (i.e., 90 00, 20D), about 70A (i.e., 100 00, 30D), about 80A (i.e., 40D), about 90A (i.e., 50D), about 100A (i.e., 60D), and/or any value between the aforementioned values. The foot cover 102 can have a top surface 110 shaped such that the foot cover 102 resembles the shape of a human foot and ankle.

As shown in FIG. 1B, the foot cover 102 can include a bottom surface 108 (e.g., a sole). The bottom surface 108 can include one or more indentations 116 (e.g., grooves, depressions, creases). In some aspects, the one or more indentations 116 can form a pattern of indentations 116. The one or more indentations 116 can extend from the toe end 112 of the bottom surface to the heel end 114 of the bottom surface 108. In some aspects, the one or more indentations 116 can extend along substantially (for example, at least over 80%, 85%, 90%, 95%, or otherwise) or entirely the bottom surface 108 (e.g., can extend across substantially an entire length and width of the bottom surface 108, an entirety of the bottom surface 108). The one or more indentations 116 can resemble wrinkled skin, or any other pattern, and can provide increased friction between the bottom surface 108 and a wet or unstable surface such as dirt or mud. In some aspects, the one or more indentations 116 can additionally include one or more cuts or micro cuts (e.g., sipes) and air bubbles that can operate to provide a suction force between the bottom surface 108 and a walking surface to facilitate gripping of the support surface by the foot cover 102.

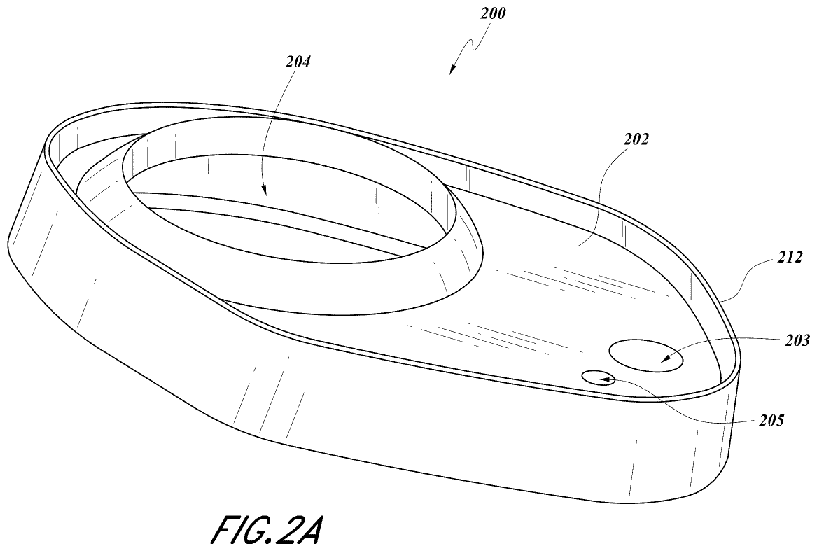

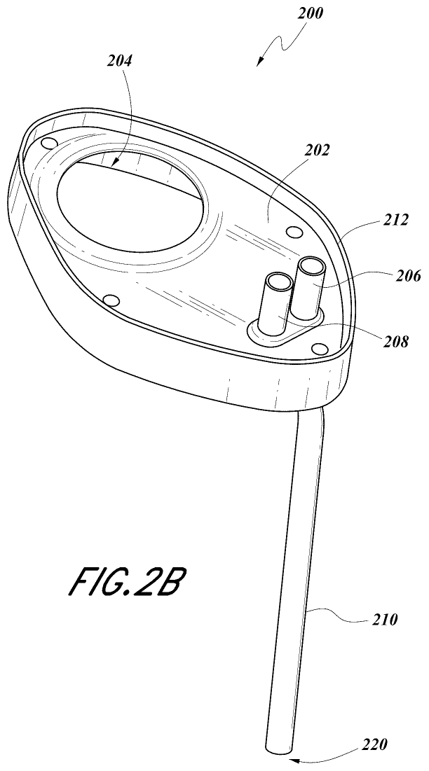

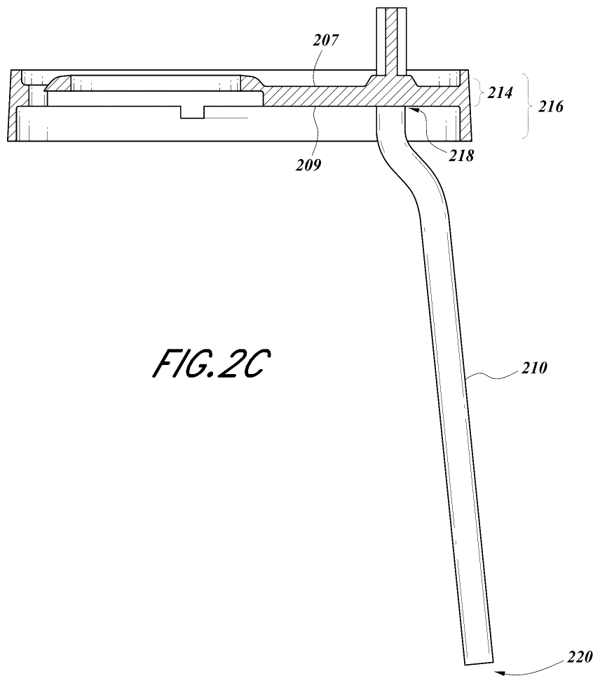

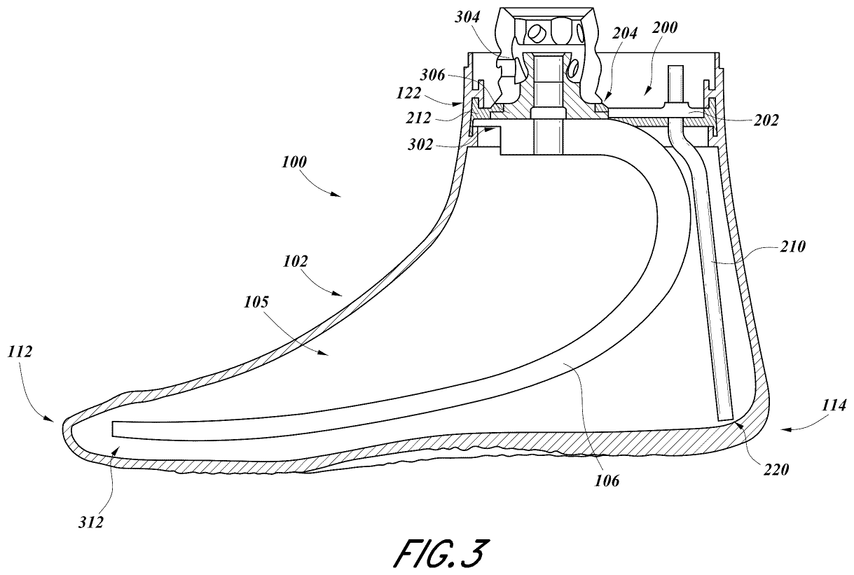

In some aspects, the foot cover 102 can include a channel 122 as shown in FIG. 1C. The channel 122 can be located on an inner surface 119 substantially near the opening 104. In some aspects, the channel 122 can border the opening 104 at a distance 121 proximate the opening 104 (for example, within a few millimeter to a few centimeter from an edge defining the opening 104). The channel 122 can include a lower lip 118 (e.g., protrusion that protrudes from the inner surface 119) and/or an upper lip 120 (e.g., protrusion that protrudes form the inner surface 119). The lower lip 118 and the upper lip 120 can form the channel 122 and can each form a recess 123 of the channel 122. In some aspects, the lower lip 118 and/or the upper lip 120 can have a stiffness. The stiffness of the lower lip 118 and/or the upper lip 120 may be greater than a stiffness of a remainder of the foot cover 102 such that the recesses 123 remain substantially the same size and shape if the foot cover 102 (for example, the opening 104 of the foot cover 102) are stretched. In some aspects, the channel 122 can secure a seal in place, for example, the seal 200 as described further below with reference to FIGS. 2A-3.

FIGS. 2A-2C illustrate an example of a seal 200 of the foot cover assembly disclosed herein. The seal 200 can include a cover portion 202 and a lip portion 212. In some aspects, the seal 200 can be substantially the same size and shape as the opening 104 such that when the seal 200 is received in the channel 122 as shown in FIG. 1C, the seal 200 forms a debris, gas and/or water-tight seal with the foot cover 102. The cover portion 202 can include a hole or aperture 204. The hole or aperture 204 can create a debris, gas and/or water-tight seal with a portion of the prosthetic foot 106 that extends through the hole or aperture 204 (see e.g., FIG. 3). In some aspects, the hole or aperture 204 can include a membrane with a waterproof, breathable material such as GORE-TEX (formerly known as expanded polytetrafluoroethylene (PTFE)).

The lip portion 212 can be coupled to the outer edge of the cover portion 202 (e.g., be one piece or monolithic with the cover portion 202). The lip portion 212 can include a thickness 216. The thickness 216 can be greater than a thickness 214 of the cover portion 202. In some aspects, the lip portion 212 can be substantially a same size and shape as the recess 123. In this way, as further described below with reference to FIG. 3, the seal 200 can be inserted into the opening 104 with the lip portion 212 coupled to (e.g., disposed in) the channel 122 so as to create a debris, gas and/or water-tight seal with the channel 122. In this way, the seal 200 inhibits (e.g., prevents) liquids, solids and/or gases from entering the inner cavity 105 of the foot cover 102.

In some aspects, the cover portion 202 of the seal 200 can include smaller holes 203, 205 than the hole or aperture 204 to accommodate a first one-way valve 206 and a second one-way valve 208. The first one-way valve 206 can, in one implementation, only allow air to flow in a first direction from above a top side 207 of the seal 200 (see FIG. 2C) through the first one-way valve 206 to below a bottom side 209 of the seal 200 (see FIG. 2C). The second one-way valve 208 can, in one implementation, only allow air to flow in a second direction from below the bottom side 209 of the seal 200 through the second one-way valve 208 to above the top side 207 of the seal 200. In some aspects, the first one-way valve 206 and/or the second one-way valve 208 are operable (e.g., selectively operable) such that air may not pass through the first one-way valve 206 and/or the second one-way valve 208 when the first and/or second one-way valves 206, 208 are closed. In some aspects, the first one-way valve 206 and/or the second one-way valve 208 can include one or more electronic switches configured to control or open the valve(s) 206, 208 when air can travel through the first one-way valve 206 and/or the second one-way valve 208. In some aspects, air that travels through the first one-way valve 206 and/or the second one-way valve 208 can cool electronic components of a prosthetic foot and/or prosthetic leg positioned inside the inner cavity 105 of the foot cover 102 and/or outside the inner cavity of the foot cover 102.

In some aspects, the first one-way valve 206 and/or the second one-way valve 208 can be configured to open when a pressure applied to the first one-way valve 206 and/or the second one-way valve 208 is at or above a pressure threshold. Accordingly, the first one-way valve 206 and/or the second one-way valve 208 can maintain a pressure inside the inner cavity 105. In some aspects, the pressure threshold may be greater than atmospheric pressure so the pressure inside the inner cavity 105 in greater than atmospheric pressure. In some aspects, the pressure threshold can include a pressure of about 0 kPa, about 1 kPa, about 2 kPa, about 3 kPa, about 4 kPa, about 5 kPa, about 6 kPa, about 7 kPa, about 8 kPa, about 9 kPa, about 10 kPa, about 11 kPa, about 12 kPa, about 13 kPa, about 14 kPa, about 15 kPa, about 20 kPa, about 25 kPa, about 30 kPa, about 35 kPa, about 40 kPa, about 45 kPa, about 50 kPa, about 60 kPa, about 70 kPa, about 80 kPa, about 90 kPa, about 100 kPa, and/or any value between the aforementioned values.

In some aspects, the first one-way valve 206 and/or the second one-way valve 208 can include a membrane with a waterproof, breathable material such as GORE-TEX (formerly known as expanded polytetrafluoroethylene (PTFE)). In this way, air can pass through the first one-way valve 206 and/or the second one-way valve 208, but the first one-way valve 206 and/or the second one-way valve 208 can prevent water, dirt and debris from passing through the first one-way valve 206 and/or the second one-way valve 208 (e.g., when the foot cover 102 is splashed with or submerged in water, such as when used on a beach).

In some aspects, as shown in FIGS. 2B and 2C, the seal 200 can include a tube 210. The tube 210 can be coupled to either the first one-way valve 206 or the second one-way valve 208 at a first end 218. In some aspects, when the seal 200 is inserted into the opening 104, the tube 210 can be positioned in the inner cavity 105 of the foot cover 102 with a free end 220 terminating inside the inner cavity 105. If the tube 210 is coupled to the first one-way valve 206, the tube may direct air flowing from outside the inner cavity 105 through the first one-way valve 206 to the second end 220 of the tube 210 and into the inner cavity 105. If the tube 210 is coupled to the second one-way valve 208, the tube 210 may direct air from the inner cavity 105 via the second end 220 through the second one-way valve 208 and out of the inner cavity 105.

FIG. 3 illustrates the foot cover assembly 100 with the prosthetic foot 106 and the seal 200 inserted through the opening 104 of the foot cover 102. The prosthetic foot 106 can include at least one foot plate. The foot plate of the prosthetic foot 106 can be inserted through the opening 104 such that a toe end 312 of a foot plate the prosthetic foot 106 is inside the inner cavity 105 substantially near (for example, within 15 mm, or within 10 mm, or within 5 mm, or otherwise, of) the toc end 112 of the foot cover 102 and an ankle end 302 of the foot plate of the prosthetic foot 106 is at substantially a same height as or near (for example, within 10 mm, or within 8 mm, or within 5 mm, or within 3 mm, or otherwise, of) the channel 122 and/or the seal 200. In some aspects, the seal 200 can be coupled to the prosthetic foot 106 before the foot plate of the prosthetic foot 106 is inserted into the cover portion 202. In other aspects, the seal 200 can be coupled to the prosthetic foot 106 after the foot plate of the prosthetic foot 106 is inserted into the cover portion 202. In some aspects, the seal 200 can be coupled to the ankle end 302 of the foot plate of the prosthetic foot 106 such that the hole or aperture 204 of the seal 200 forms a debris, gas and/or water-tight seal with a bottom adaptor 306 of the prosthetic foot 106. In some aspects, a top adaptor 304 of the prosthetic foot 106 can be coupled to the bottom adaptor 306 such that the seal 200 is secured to the prosthetic foot 106. As described above with reference to FIGS. 2A-2C, when the prosthetic foot 106 and the seal 200 are inserted in the foot cover 102, the lip portion 212 of the seal 200 can be coupled to (e.g., disposed or positioned in) the channel 122 (e.g., retained between the upper lip 120 and the lower lip 118). The seal 200 can secure the prosthetic foot 106 in place such that the foot plate of the prosthetic foot 106 stays in place inside the inner cavity 105. In some aspects, a calf or leg cosmesis or any other cosmesis can be coupled to the seal 200 and/or the foot cover 102.

In some aspects, when the seal 200 is coupled to the channel 122, the free end 220 of the tube 210 can be substantially near the heel end 114 of a bottom portion of the inner surface 119 (for example, within a few millimeters). In some aspects, if the tube 210 is coupled to the first one-way valve 206, air flowing through the first one-way valve 206 into the inner cavity 105 can be used to pressurize the inner cavity 105. In this way, pressurized air can stretch the foot cover 102, inhibiting (e.g., reducing, preventing) possible wrinkles in the foot cover 102, which can lead to cracks in the foot cover 102. The pressurized air and stretched cover 102 can also assist with or resist bending of the foot cover 102, or the pressurized air and stretched foot cover 102 can provide a dampening effect in order to protect the prosthetic foot 106.

In some aspects, if the tube 210 is coupled to the second one-way valve 208, the tube 210 can draw air out of the inner cavity 105 creating a partial vacuum or vacuum effect in the inner cavity 105. The partial vacuum or vacuum effect can shrink the foot cover 102 and create a tighter fit between the inner cavity 105 and the foot cover 102 reducing an amount of movement of the prosthetic foot 106 inside the foot cover 102.

In some aspects, the foot cover 102 can include one or more portions with a support. The support can be a textile or other material coupled to the inner surface 119. The support can reduce an amount of stretching or shrinking at the one or more portions of the inner surface 119 in order to maintain a shape of the foot cover 102 while changing (for example, increasing or decreasing) a pressure inside the inner cavity 105. In some aspects, the support can stretch in one direction. For example, the support can stretch in a first direction (e.g., up and down in the direction of the height of the foot cover 102), but not in a second direction perpendicular to the first direction. In some aspects, the support can flex or bend, but not stretch. In this way, the support can allow the foot cover 102 to flex, however, the support can prevent the foot cover 102 from stretching.

In some aspects, air may travel through the first one-way valve 206 and/or the second one-way valve 208 due to changes in pressure inside the inner cavity 105. For example, as the user takes a step and the bottom surface 108 of the foot cover 102 contacts a walking surface, a force may be applied to the bottom surface 108, increasing the pressure in the inner cavity 105 and forcing air through the second one-way valve 208. In some aspects, air travelling through the first one-way valve 206 and/or the second one-way valve 208 can optionally cool a connection between a prosthetic foot and a limb of a user. In some aspects, the air travelling through the first one-way valve 206 and/or the second one-way valve 208 can optionally cool electronics or other components of the prosthetic foot 106 or a prosthetic leg coupled to the prosthetic foot.

In some aspects, a vacuum bag and/or a filter can be coupled to the free end 220 of the tube 210. The prosthetic foot 106 can include multiple parts. Friction between the multiple parts can cause debris to build up in the inner cavity 105. Air travelling through the second one-way valve 208 can create a suction effect at the free end 220 of the tube 210 forcing the debris into the vacuum bag. The filter can be coupled to the free end 220 such that debris does not enter the tube 210 and damage the second one-way valve 208.

In some aspects, air travelling through the first one-way valve 206 and/or the second one-way valve 208 can provide power (e.g., mechanical power) to move one or more levers of the prosthetic foot 106. The one or more electronic switches of the first one-way valve 206 and/or the second one-way valve 208 can control the air flowing in and out of the inner cavity 105, and the tube 210 may direct the air to a desired component of the prosthetic foot 106. Airflow from the free end 220 of the tube 210 can move the one or more levers and/or alter other mechanical parts of the prosthetic foot 106 in order to change a geometry of the prosthetic foot 106 and/or alter a stiffness of a portion of the prosthetic foot 106.

In some aspects, the airflow through the first one-way valve 206 and/or the second one-way valve 208 can be converted into electricity. The airflow can apply a pressure to a piezoelectric material, deforming the piezoelectric material and generating electricity that can be used to power components of the prosthetic foot 106 and/or any other prosthetic limb attached to the prosthetic foot 106. In some aspects, the piezoelectric material can be coupled to a portion of the prosthetic foot 106. When a user walks, the at least one foot plate of the prosthetic foot 106 may flex inside the inner cavity 105 deforming the piezoelectric material.

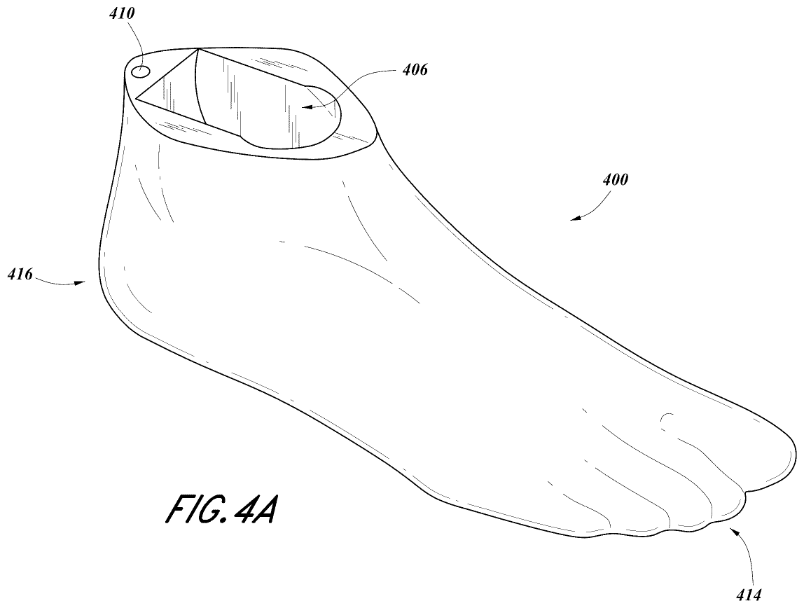

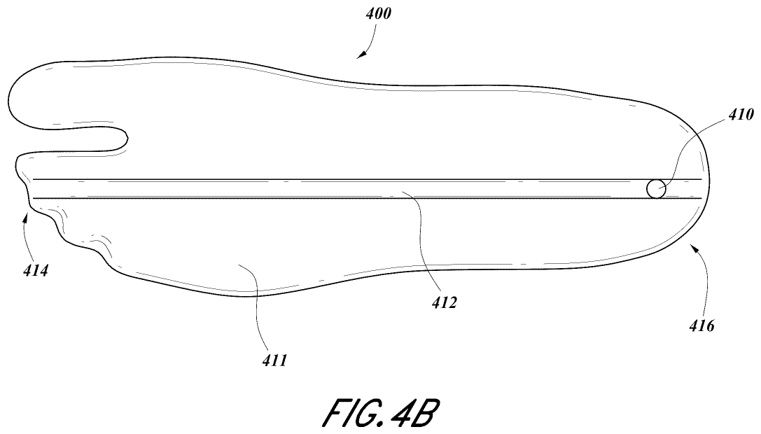

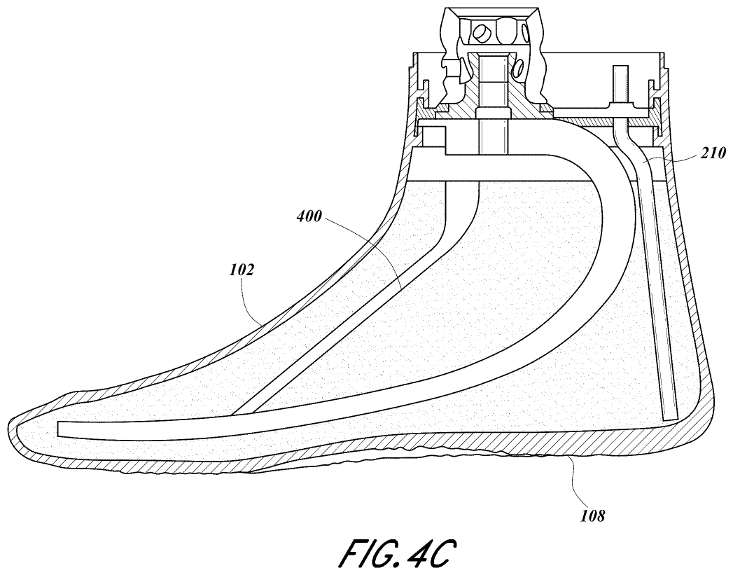

As shown in FIGS. 4A-4C, the foot cover assembly 100 can include an inner shell 400 (e.g., substructure). The inner shell 400 can be configured to be inserted into the inner cavity 105. The substructure can be substantially the same size and shape as the inner cavity 105 such that when the inner shell 400 is inserted in the inner cavity 105, the inner shell 400 does not move relative to the inner cavity 105. In some aspects, the inner shell 400 and the inner cavity 105 can resemble the shape of a human foot. The inner shell 400 can be made of at least one of foam, polycarbonate, polyethylene, polyvinyl chloride, ethylene-vinyl acetate, thermoplastic polyurethane, expanded thermoplastic polyurethane, injected rubber, compressed rubber or any other stiff material. In some aspects, the inner shell 400 can be 3D printed (e.g., via an additive manufacturing process). In some aspects, if the foot cover 102 is a flexible material, the inner shell 400 can provide support to the foot cover 102 so the foot cover 102 does not deform during use.

In some aspects, the inner shell 400 can include a foot cavity 406. The foot cavity 406 can be substantially the same shape as the prosthetic foot 106 and the foot cavity 406 can be configured to receive at least a portion of the prosthetic foot 106. In some aspects, the prosthetic foot 106 can be inserted into the foot cavity 406 before the prosthetic foot 106 and the inner shell 400 are inserted into the foot cover 102. In other aspects, the at least one foot plate of the prosthetic foot 106 can be inserted into the inner shell 400 after the inner shell 400 is inserted into the inner cavity 105. The inner shell 400 can be configured such that when both the inner shell 400 and the prosthetic foot 106 are inserted in the inner cavity 105 of the foot cover 102, the inner shell 400 holds the prosthetic foot 106 in place such that the prosthetic foot 106 is inhibited from (e.g., prevented from) moving around in the inner cavity 105. In some aspects, the inner shell 400 can protect the prosthetic foot 106 such that forces applied to the prosthetic foot 106 during walking or running are reduced.

The prosthetic foot 106 can be secured in the foot cavity 406 via adhesive. The adhesive can be applied to the prosthetic foot 106 and/or the foot cavity 406. The adhesive can be cured while the prosthetic foot 106 is positioned in the foot cavity 406. A pressure or force can be applied to the prosthetic foot 106 and/or the inner shell 400 while the adhesive cures. In some aspects, heat can be applied to the prosthetic foot 106, the inner shell 400, and/or the adhesive to reduce or minimize a time required for the adhesive to cure.

In some aspects, the inner shell 400 can include a tube cavity 410. The tube cavity 410 can be substantially the same size and shape as the tube 210 such that the tube 210 can be inserted into the tube cavity 410. In this way, the inner shell 400 can provide support to the tube 210 so the tube 210 does not bend or break when the foot cover assembly 100 is in use. The inner shell 400 can also keep the tube 210 in place when the foot cover assembly 100 is in use. In some aspects, the inner shell 400 can include a bottom channel 412 connected to the tube cavity 410 on a bottom surface 411 of the inner shell 400. The bottom channel 412 can extend from a toe end 414 to a heel end 416 of the inner shell 400. The bottom channel 412 can provide a gap between the inner shell 400 and a bottom portion of the inner surface 119 of the foot cover 102 such that air in the bottom channel 412 can be drawn out of the inner cavity 105 through the tube 210, or air that enters the inner cavity 105 through the tube 210 can flow into the bottom channel 412. In this way, if the inner shell 400 is in the inner cavity 105 of the foot cover 102, the first one-way valve 206 and second one-way valve 208 can still be used pressurize the inner cavity 105 or remove air from the inner cavity 105 through the tube 210. In some aspects, the inner shell 400 can include more than one bottom channel 412. In some aspects, the bottom channel 412 can be an open cell structure of a 3D printed inner shell 400.

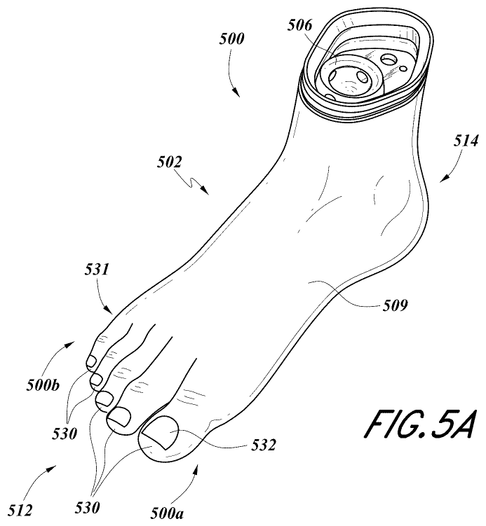

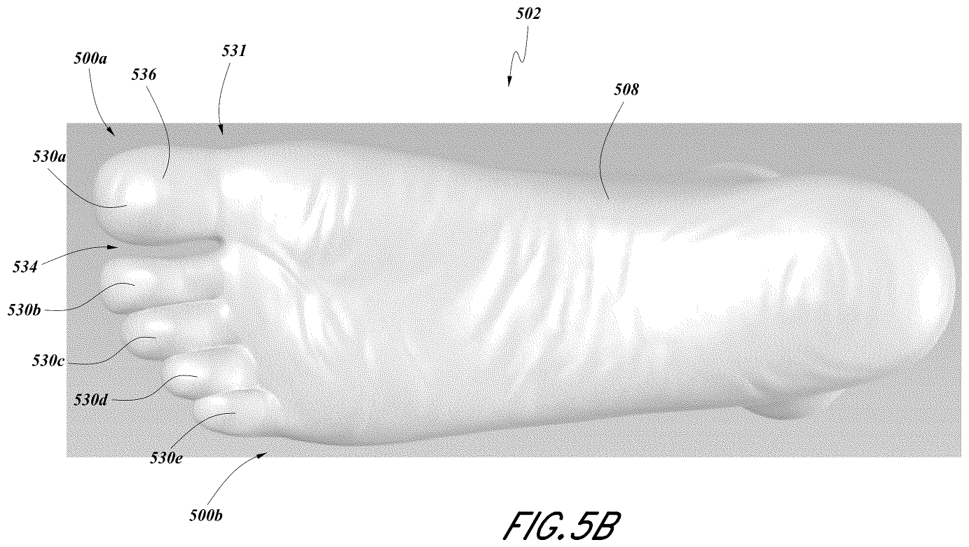

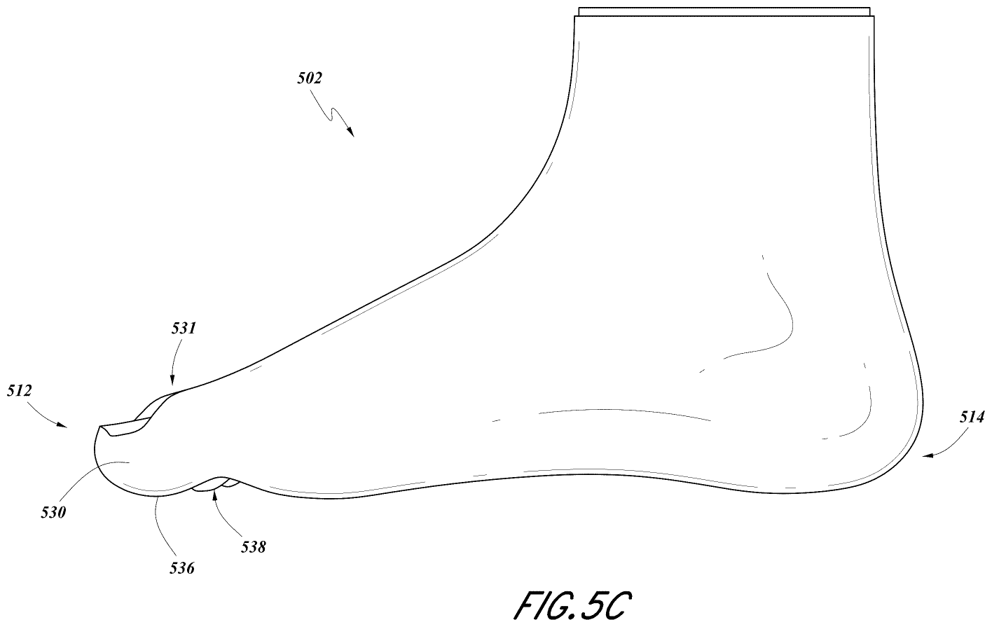

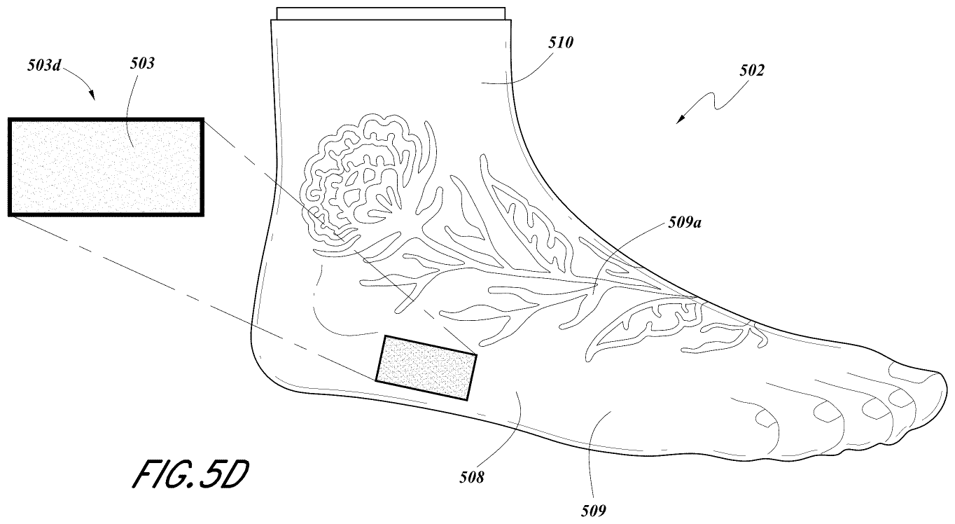

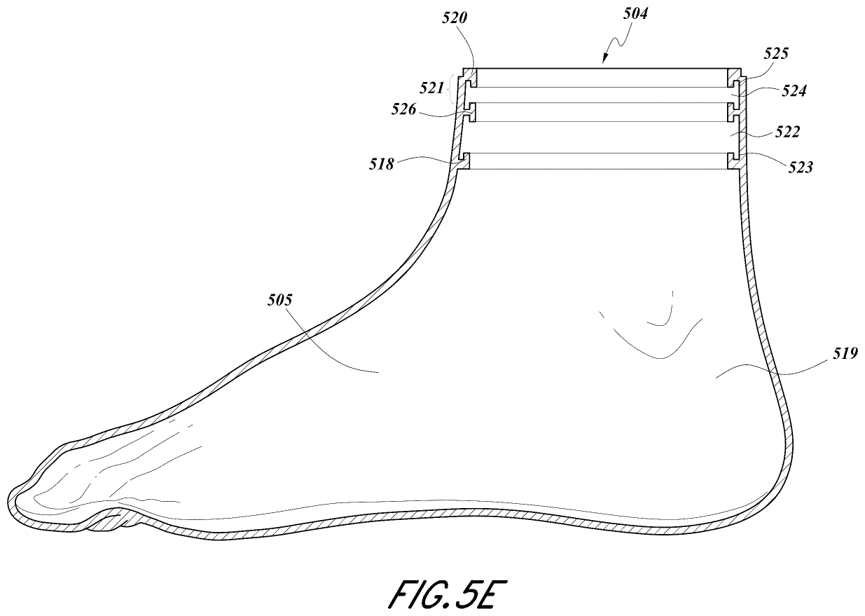

FIGS. 5A-5E illustrate an example foot cover assembly 500. The foot cover assembly 100 in FIGS. 1A-4C can have any of the features of the foot cover assembly 500, and the foot cover assembly 500 can have any of the features of the foot cover assembly 100 except for the differences described below. As shown in FIG. 5A, the foot cover assembly 500 can extend from a toe end 512 to a heel end 514. The foot cover assembly 500 includes a foot cover 502 that can extend between the toc end 512 and the heel end 514.

The foot cover 502 can include an outer surface 509. The outer surface 509 can include toes 530 at the toe end 512 of the foot cover 502. The outer surface 509 can be shaped such that the toe end 512 includes toes 530 that resemble the shape of human toes. The toes 530 can extend between the toe end 512 of the foot cover 502 to a second end 531. The second end 531 can include an end of the toes 530 opposite the toe end 512 of the foot cover 502 that is connected to the remainder of the foot cover 502.

Each toc 530 can include a nail 532. The nail 532 can be shaped such that the nail 532 resembles a human toenail. A portion of the outer surface 509 that includes the nail 532 can be polished or otherwise altered so the nail 532 includes a glossy, shiny, or otherwise nail like appearance. In some aspects, the portion of the outer surface 509 that includes the nail 532 can be polished or otherwise altered using a polishing tool. In some aspects, a portion of a molding tool (e.g., an injection mold) positioned at a position of the nail 532 can include a polished surface. The nail 532 can be configured to receive and/or be coupled to an artificial nail. The nail 532 can be shaped to allow artificial nails to be coupled or bonded to the portion of the outer surface 509 that include the nail 532.

As shown in FIG. 5B, the foot cover 502 can include a first toe 530A, a second toc 530B, a third toe 530C, a fourth toe 530D, and/or a fifth toe 530E. The first toe 530A can be positioned at on a medial side 500A of the foot cover assembly 500 and the fifth toe 530E can be positioned on a lateral side 500B the foot cover assembly 500. The second toe 530B can be positioned laterally to the first toe 530A, the third toe 530C can be positioned laterally to the second toe 530B, the fourth toe 530D can be positioned laterally to the third toe 530C, and/or the fifth toe 530E can be positioned laterally to the fourth toe 530D. The foot cover 502 can include a gap 534 between the first toe 530A and the second toe 530B. The gap 534 may be configured to receive a portion of a strap (i.e., a toe post) of footwear (e.g., sandal, flip-flop, etc.).

As shown in FIG. 5C, a bottom side 536 of the toes 530 at the second end 531 of the toes 530 can form a recess 538. The toes 530 can be shaped so the bottom side 536 of the toes 530 at the second end 531 of the toes 530 is raised from the rest of the toes 530 and/or a bottom surface 508 of the foot cover 502 between the second end 531 and the heel end 514. The recess 538 can be configured to engage or contact a raised surface or ridge of footwear, and/or a raised surface/ridge coupled to the footwear so the recess 538 and/or the foot cover 502 can grip, lock, or otherwise secure the foot cover 502 to the footwear.

As shown in FIG. 5D, the outer surface 509 of the foot cover 502 can include a pattern or a texture 503. Inset image 503D shows details of the pattern or texture 503 of the outer surface 509. Additionally, a bottom surface 508 of the foot cover 502 and/or a top surface 510 of the foot cover 502 can include the texture 503. The texture 503 can be configured to resemble skin (for example, human skin, pig skin, and/or the like) or any other patterns or texture. The texture 503 can increase grip or friction between the outer surface 509 of the foot cover 502 and a user, and/or the outer surface 509 and a walking surface and/or reduce or minimize visibility of parting lines, scratches, surface faults, or other wear on the outer surface 509. The texture 503 can reduce or minimize a surface tension of the outer surface 509 and/or reduce wear of a portion of the outer surface 509. Accordingly, a flex life located where the foot cover 502 flexes during use can be increased. In some aspects, the texture 503 can allow ink or dyes to bond or bind to the outer surface 509. Accordingly, permanent and/or semi-permanent images or patterns 509A can be applied to the outer surface 509, for example, henna, tattoos, etc. The images or patterns 509A can be used to reduce or minimize visibility of parting lines, scratches, surface faults, or other wear on the outer surface 509.

In some aspects, the foot cover 502 (for example, at least the outer surface 509 of the foot cover 502) can be colored to resemble a skin color of a user. The skin color of the user can be determined using skin color chart or matching system (e.g., a cosmetics skin color chart, the Pantone color system, etc.), images or the user's skin, and/or one or more computer application configured to determine color data of an image. In order to color the foot cover 502 (for example, the outer surface 509), a dye or ink can be applied to the foot cover 502 (for example, the outer surface 509), and/or the dye or ink can be boiled onto, into, or through the foot cover 502 (for example, the outer surface 509).

As shown in FIG. 5E, the foot cover 502 can include a first channel 522 and a second channel 524. The first channel 522 and the second channel 524 can be located on an inner surface 519 of the foot cover 502 substantially near (for example, within a few millimeters or within a few centimeters of) an opening 504 of an inner cavity 505 of the foot cover 502. The second channel 524 can border the opening 504 and the second channel 524 can be positioned a distance 521 (for example, about 10 mm, or about 5 mm, or about 3 mm, or otherwise) proximate the opening 504. The foot cover 502 can include a lower lip 518 (e.g., protrusion that protrudes from the inner surface 519), an upper lip 520 (e.g., protrusion that protrudes form the inner surface 119), and/or a middle lip 526 (e.g., protrusion that protrudes form the inner surface 119). The middle lip 526 can be positioned between the lower lip 518 and the upper lip 520. The lower lip 518 and the middle lip 526 can form the first channel 522. The upper lip 520 and the middle lip 526 can form the second channel 524. The lower lip 518 can form a recess 523 of the first channel 522 and the upper lip 520 can form a recess 525 of the second channel 524. The middle lip 526 can form the recess 523 of the first channel 522 and the recess 525 of the second channel 524.

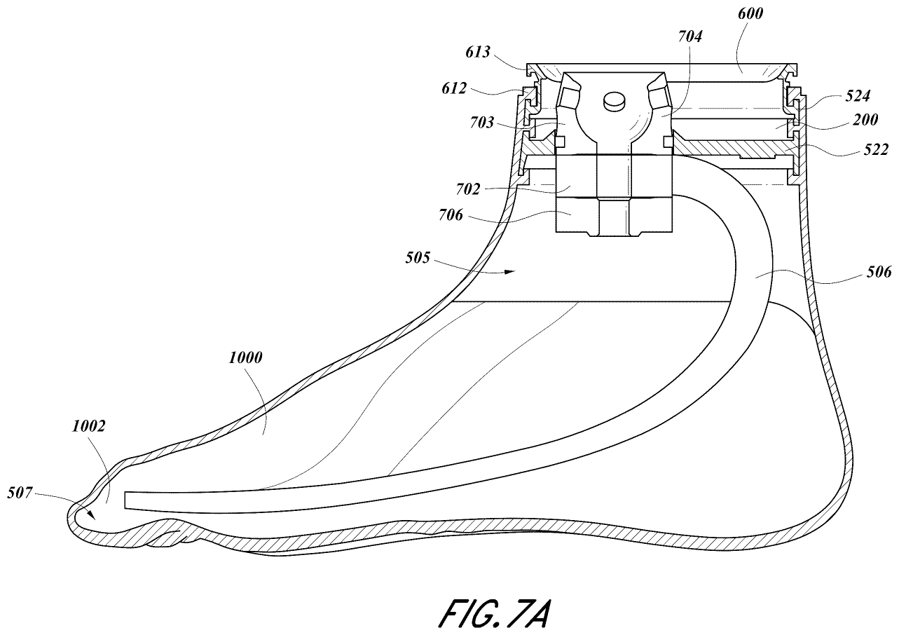

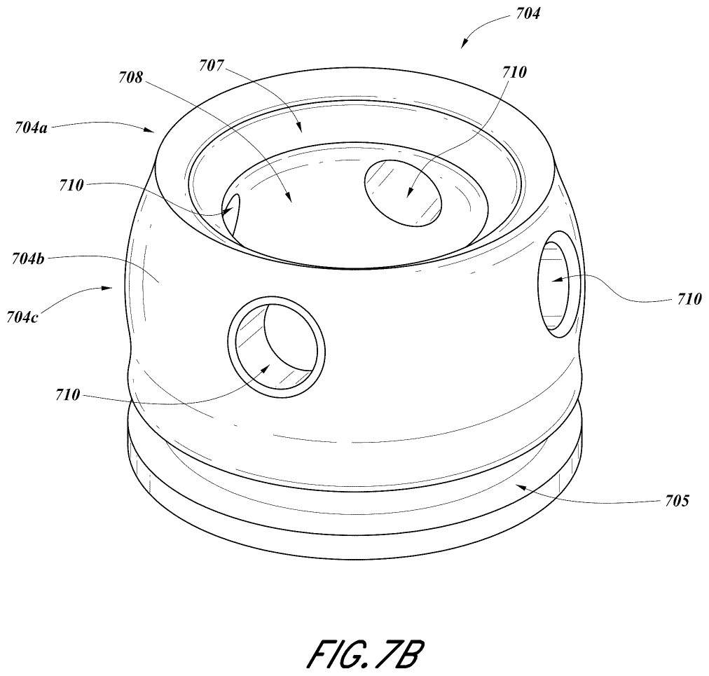

In some aspects, the lower lip 518, the upper lip 520, and/or the middle lip 526 can have a stiffness. The stiffness of the lower lip 518, the upper lip 520, and/or the middle lip 526 can be greater than a stiffness of the remainder of an upper wall of the foot cover 502 such that the recesses 523, 525 remain substantially the same size and shape if the foot cover 502 (for example, the opening 504) is stretched. In some aspects, as shown in FIG. 7, the first channel 522 can secure the seal 200 disclosed herein in place and the second channel 524 can secure a retainer 600 in place.

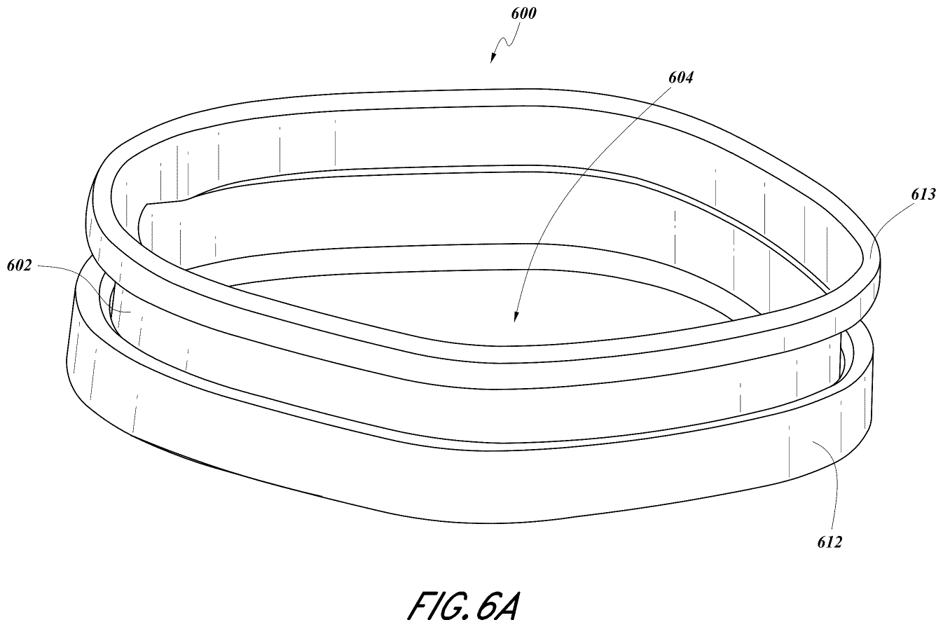

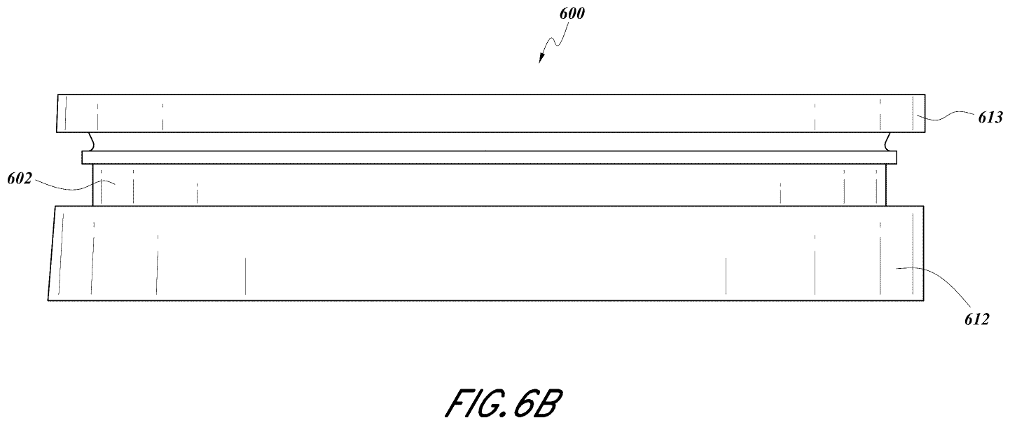

FIGS. 6A and 6B illustrate an example retainer 600. The retainer 600 can include a ring-shaped sidewall 602 substantially the same size and shape as the opening 504. The sidewall 602 can form a hole 604. The hole 604 can allow a user to access to a prosthetic foot 506 and/or the seal 200 when the retainer 600 is coupled to the foot cover 502, as shown in FIG. 7. The retainer 600 can include a first lip 612 and/or a second lip 613. The first lip 612 and/or the second lip 614 can extend from an outer surface 603 of the sidewall 602. The first lip 612 can be substantially a same size and shape as the recess 525 of the second channel 522. Accordingly, as shown in FIG. 7, the retainer 600 can be inserted into the opening 504 with the first lip 612 coupled to (e.g., disposed in) the second channel 524. The first lip 612 can create a debris, gas and/or water-tight seal with the second channel 524.

FIG. 7A illustrates the foot cover assembly 500 with the prosthetic foot 506, the seal 200, and the retainer 600 inserted through the opening 504 of the foot cover 502. An adaptor 703 can be coupled to an ankle end portion 702 of a foot plate of the prosthetic foot 506. A top portion 704 of the adaptor 703 can be positioned above the ankle end portion 702 of the foot plate of the prosthetic foot 506 and a bottom portion 706 of the adaptor 703 can be positioned below the ankle end portion 702. The top portion 704 of the adaptor 703 can be coupled to the bottom portion 706 via a fastener (e.g., a screw, bolt, etc.). When the prosthetic foot 506 is positioned in the inner cavity 505 of the foot cover 502, the top portion 704 of the adaptor 703 can extend through the hole or aperture 204 of the seal 200.

FIG. 7B illustrates the top portion 704 of the adaptor 703. In some aspects, the top portion 704 of the adaptor 703 can include a male pyramid adaptor. The top portion 704 of the adaptor 703 can include a groove 705. In some aspects, the groove 705 may be positioned below a dome portion 704C of the top portion 704. The groove 705 can be configured to receive a gasket (e.g., an x-ring, an O-ring, etc.). The gasket can form a debris, gas and/or water-tight seal between the top portion 704 of the adaptor 703 and the seal 200. The top portion 704 of the adaptor 703 can be configured to couple the prosthetic foot 506 to a prosthetic leg. The top portion 704 can include a recess 708 and an opening 707 to the recess 708 at a top end 704A of the top portion 704. The recess 708 may be configured to receive a portion of a prosthetic leg (e.g., an adaptor) via the opening 707. In some aspects, the recess 708 may be generally spherically shaped or include a curved inner surface.

The top portion 704 of the adaptor 703 can include holes 710 extending from an outer surface 704B of the top portion 704 to the recess 708. The holes 710 can be configured to receive a fastener (e.g., a screw, a bolt, etc.). The holes 710 can include threading configured to interact with threading of the fastener. In some aspects, the threading of the holes 710 can include fine threading or coarse threading. The threading of the holes 710 can include an outer diameter of about 1 mm (M1), about 2 mm (M2), about 3 mm (M3), about 4 mm (M4), about 5 mm (M5), about 6 mm (M6), about 7 mm (M7), about 8 mm (M8), about 9 mm (M9), about 10 mm (M10), about 12 mm (M12), about 14 mm (M14), about 16 mm (M16), and/or any value between the aforementioned values. The fastener can be configured to secure the portion of the prosthetic leg in the recess 708 (i.e., prevent or inhibit the portion of the prosthetic leg from moving out of the recess 708). In some aspects, when the fastener is positioned in the holes 710, the fastener can be rotated in a first direction to translate the fastener towards the recess 708 (i.e., into the holes 710), and the fastener can be rotated in a second direction opposite the first direction to translate away from the recess 708 (i.e., out of the holes 710).

In some aspects, the top portion 704 can include a plurality of holes 710. The top portion 704 can include two holes 710, three holes 710, four holes 710, five holes 710, and/or six holes 710. The plurality of holes 710 can be circumferentially spaced around the top portion 704 of the adaptor 703.

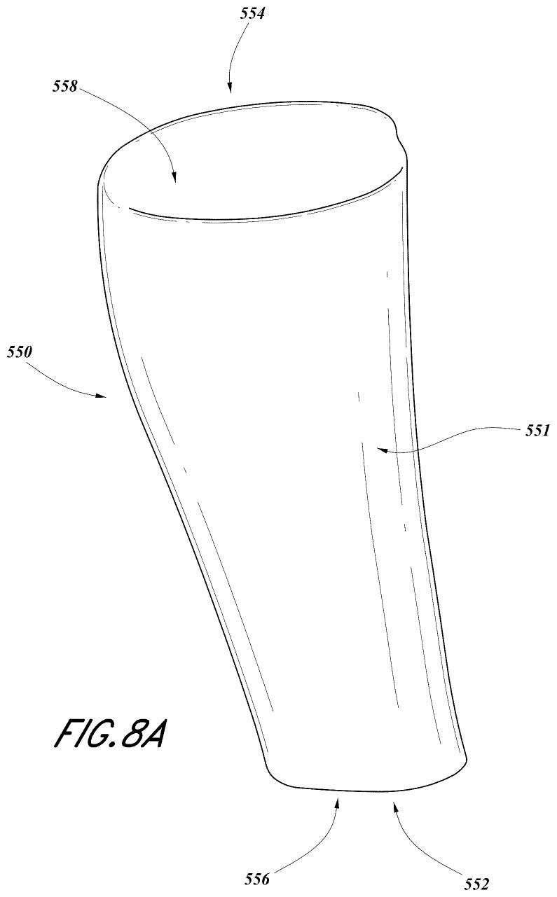

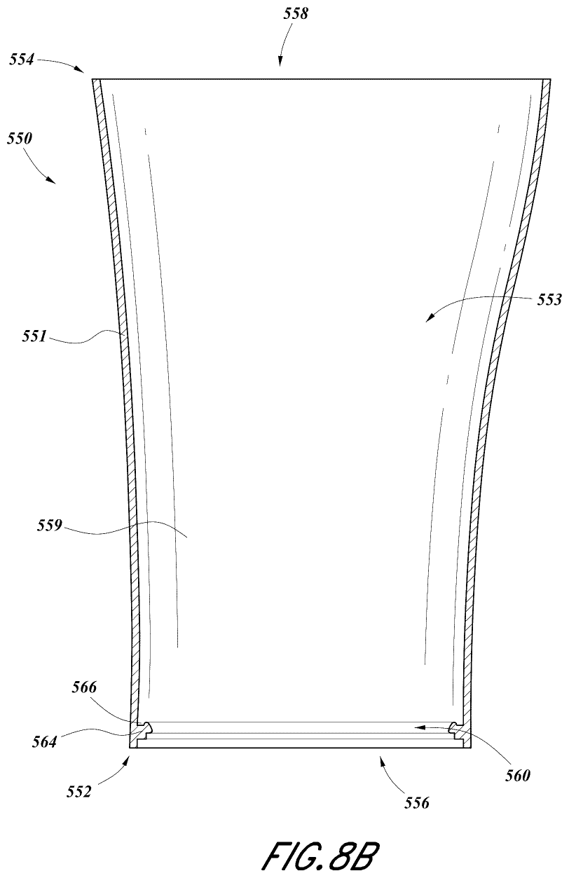

FIGS. 8A and 8B illustrate an example of a leg cosmesis 550. The leg cosmesis 550 can be configured to cover a prosthetic leg, or a portion thereof, coupled to the prosthetic foot 506. The leg cosmesis 550 can extend from an ankle end 552 to a top end 554 opposite the ankle end 552. The leg cosmesis 550 can include a cover 551. The cover 551 can be shaped such that the cover 551 resembles the shape of a human leg or a portion thereof. The cover 551 can include a first opening 556 at the ankle end 552 and/or a second opening 558 at the top end 554. The cover 551 can include an inner cavity 553 extending from the first opening 556 to the second opening 558. A prosthetic leg can be inserted through the inner cavity 553 via the first opening 556 and/or the second opening 558, as shown in FIG. 8B. The cover 551 can be made of rubber, silicone, nitrile, polyvinyl chloride, neoprene, or any other water-resistant rubber replacement material.

The cover 551 can include a channel 560 as shown in FIG. 8B. The channel 560 can be located on an inner surface 559 of the cover 551 at or substantially near the first opening 556. In some aspects, the channel 560 can border the first opening 556 at a distance 562 (for example, about 10 mm, or about 5 mm, or about 3 mm, or otherwise,) proximate the first opening 556. The channel 560 can include a lip 564 (e.g., protrusion that protrudes from the inner surface 559). The lip 564 can form the channel 560 and a recess 566 of the channel 560. In some aspects, the lip 564 can have a stiffness. The stiffness of the lip 564 may be greater than a stiffness of a remainder of the cover 551 such that the recess 566 remains substantially the same size and shape if the cover 551 (for example, the first opening 556) is stretched.

The retainer 600 can be configured to be at least partially inserted into the leg cosmesis 550 to couple the leg cosmesis 550 to the foot cover assembly 500. The second lip 613 of the retainer 600 can be substantially a same size and shape as the recess 566 of the channel 560. Accordingly, the retainer 600 can be inserted into the first opening 556 and/or the cover 551 can be placed over the retainer 600 with the second lip 613 coupled to (e.g., disposed in) the channel 560. The second lip 613 can create a debris, gas and/or water-tight seal with the channel 560. Accordingly, the retainer 600 can create a debris, gas and/or water-tight seal between the cover 551 of the leg cosmesis 550 and the foot cover 502 of the foot cover assembly 500.

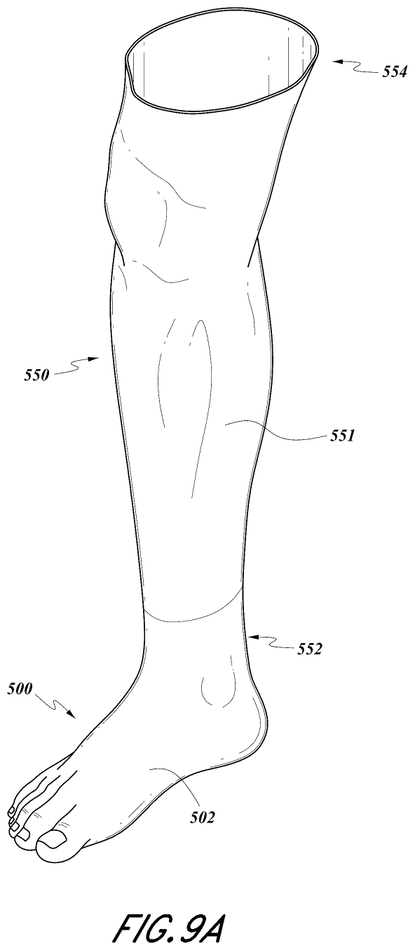

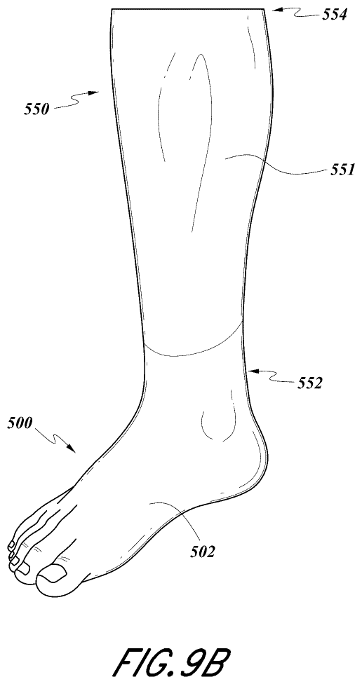

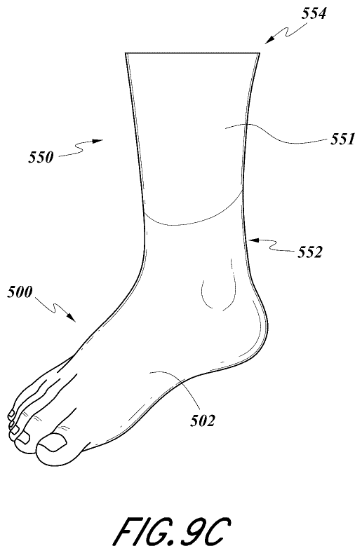

FIGS. 9A-9C illustrate the foot cover assembly 500 with examples of the leg cosmesis 550 of various sizes and shapes coupled to the foot cover assembly 500. As shown in FIG. 9A, in some aspects, the cover 551 can be shaped such that the cover 551 resembles the shape of a portion of a human leg extending from an ankle of the human leg (i.e., the ankle end 552 of the cover 551) to a position above a knee of the human leg (i.e., the top end 554 of the cover 551). Accordingly, the leg cosmesis 550 can be configured to cover a portion of a prosthetic leg between the foot cover 502 and a position above a knee of the prosthetic leg. As shown in FIGS. 9B and 9C, in some aspects, the cover 551 can be shaped such that the cover 551 resembles the shape of a shin/calf (FIG. 9B), or a portion thereof (FIG. 9C), of the human leg extending from the ankle of the human leg (i.e., the ankle end 552 of the cover 551) to a position below the knee of the human leg (i.e., the top end 554 of the cover 551). Accordingly, the leg cosmesis 550 can be configured to cover a portion of a prosthetic leg between the foot cover 502 and a position below the knee of the prosthetic leg.

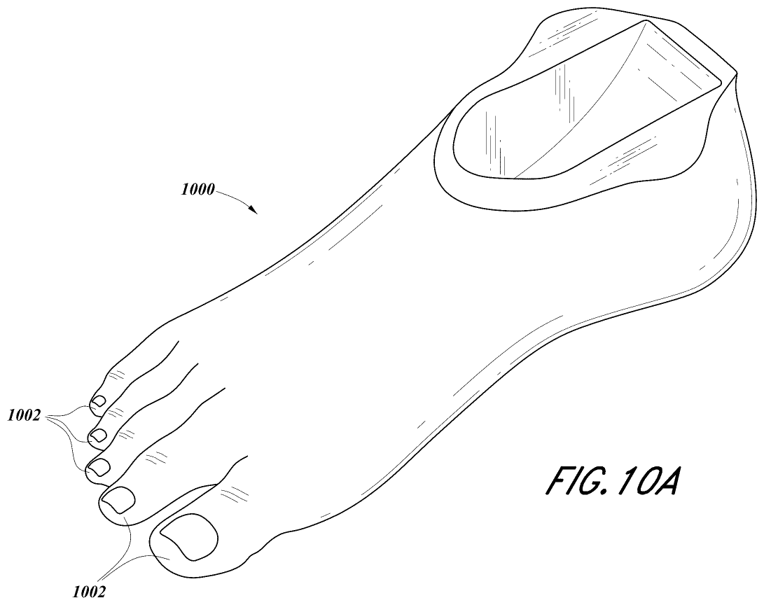

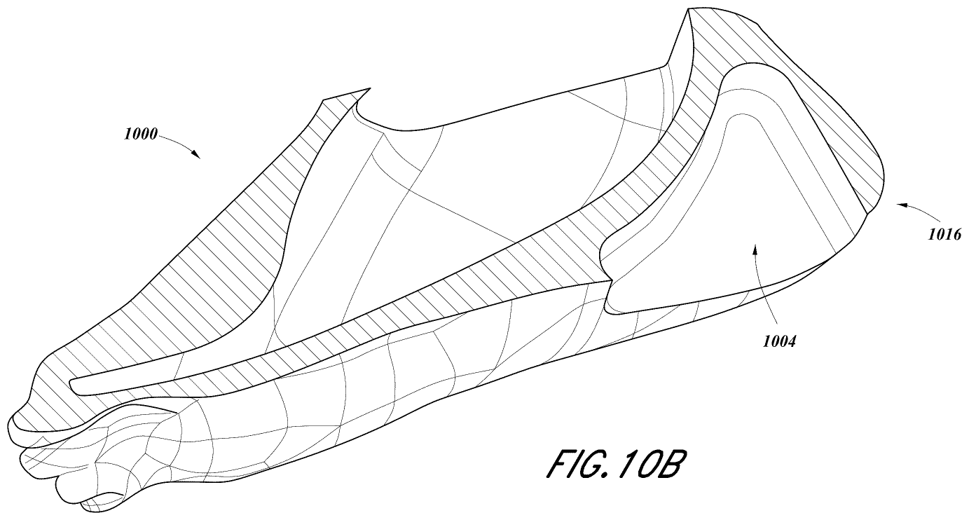

FIGS. 10A and 10B illustrate an inner shell 1000 (e.g., substructure). The inner shell 400 in FIGS. 4A-4C can have any of the features of the inner shell 1000, and the inner shell 1000 can have any of the features of the inner shell 400 except the differences described with reference to FIGS. 10A and 10B. As shown in FIG. 7, the inner shell 1000 can be configured to be inserted into the inner cavity 505 of the foot cover 502. In some aspects, the inner shell 1000 and the inner cavity 505 can include a same shape as the outer surface 509 of the foot cover 502 (i.e., a human foot) so the inner shell 1000 and the inner cavity 505 include toes 1002 and 507, as shown in FIGS. 5E and 7. The toes 1002 of the inner shell 1000 can allow the inner shell 1000 to flex similar to a human foot during a human gait cycle, such as when transitioning from a midstance phase to a push off phase, so the inner shell 1000 and thus the foot cover assembly 500 can better approximate the functions of a human foot.

As shown in FIG. 10B, the inner shell 1000 can include a recess 1004 extending from a bottom surface 1011 of the inner shell 1000. The recess 1004 can be positioned at a heel end 1016 of the inner shell 1000. The recess 1004 can be configured to receive an insert the same size and shape as the recess 1004. The insert can include a different material from the inner shell 1000 so the inner shell 1000 and the insert have a different stiffness and/or density. For example, the inner shell 1000 can comprise foam, and the insert can comprise compressed rubber. In some aspects, the insert and/or the material of the insert can be stiffer (i.e., more rigid) than the inner shell 1000 and/or the material of the inner shell 1000. In some aspects, the stiffness and/or the density of the insert can vary throughout the insert. For example, a medial portion of the insert can include a different stiffness and/or density than a lateral portion of the insert, and/or an anterior portion of the insert can include a different stiffness and/or density than a posterior portion of the insert. The material, the stiffness, and/or the density of the insert can be selected based on a weight or mass of a user. Therefore, the inner shell 1000 can be produced or manufactured using one material for users of different weights to simplify manufacturing and/or reduce manufacturing costs. If a weight or mass of the user changes, the insert can be removed from the recess, and a second insert with a material, stiffness and/or density that is different from the insert can be inserted into the recess 1004 to accommodate the change in weight or mass.

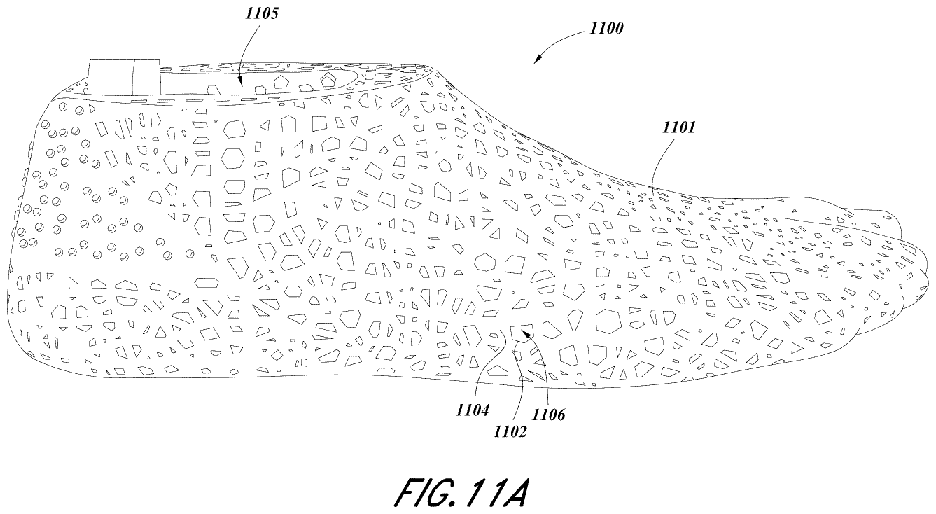

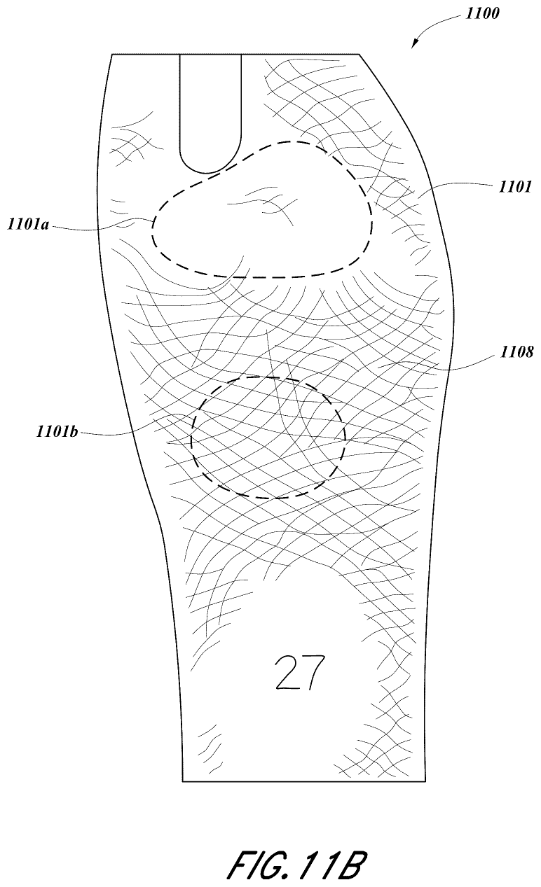

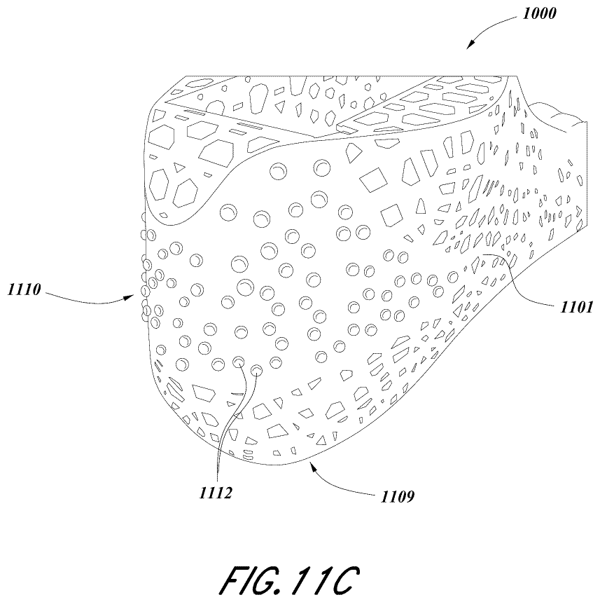

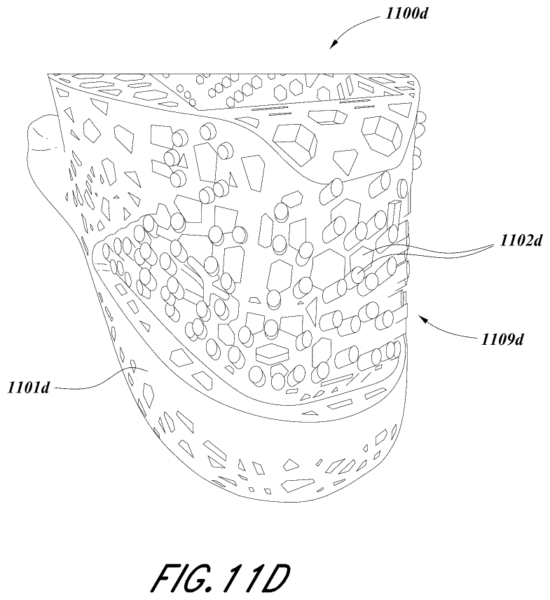

FIGS. 11A-11C illustrate an example inner shell 1100. The inner shell 400, 1000 in FIGS. 4A-4C and 10A-10B can have any of the features of the inner shell 1100 and the shell 1100 can have any of the features of the foot covers 400, 1000 except the differences described with reference to FIGS. 11A-11D. The inner shell 1100 can be 3D printed (e.g., via an additive manufacturing process). As shown in FIG. 11A, the inner shell 1100 can include a body 1101 and a foot cavity 1105 in the body 1101. The foot cavity 1105 can be configured to receive a prosthetic foot, such as the prosthetic foot examples disclosed herein or a different prosthetic foot. The body 1101 can include a plurality of interconnected cells 1102 (i.e., an open cell structure). Each cell 1102 can include struts 1104 that define an opening 1106.

As shown in FIG. 11B, a cell density (i.e., the number of cells 1102 per unit of volume) can vary throughout the body 1101 of inner shell 1100. Additionally, the size and shape of the struts 1104 and/or the opening 1106 of each cell 1102 can vary depending on a position of each cell 1102 on the body 1101. Accordingly, a stiffness of the body 1101 can vary throughout the body 1101. For example, a portion 1101A of the body 1101 with a high cell density (i.e., a high number of cells 1102 or cells 1102 with small openings) can include a higher stiffness (i.e., more rigid) than a portion 1101B of the body 1101 with a low cell density (i.e., a low number of cells 1102 or cells 1102 with large openings).

The body 1101 can approximate the varying stiffness of bones, tendons, and/or soft tissues throughout a human foot. Accordingly, the body 1101 can flex similar to a human foot during a human gait cycle, such as when transitioning from a midstance phase to a push off phase, so the body 1101 can better approximate the anatomy, the stiffness, and/or the functions of a human foot. For example, the body 1101 can approximate the rollover of a human foot during the human gait cycle. In some aspects, the cell density of each portion of the body 1101 can be selected based on an anatomy of a foot and/or a gait cycle of a particular user so the body 1101 can approximate the anatomy, the stiffness, and/or the functions of a particular user's foot. One or more tests may be performed to determine the stiffness of the user's foot at various locations and the determined stiffness of the user's foot can be used to select the cell density and/or the stiffness of each portion of the body 1101.

In some aspects, the plurality of interconnected cells 1102 can flex, expand, and/or compress so the body 1101 can flex, expand, and/or compress similar to a human foot. As the user takes a step and a bottom surface 1108 contacts a walking surface, a force (i.e., pressure) may be applied to the bottom surface 1108, each cell 1102 of the plurality of interconnected cells 1102 can flex, expand, and/or compress so a width 1108A and/or a length 1108B of the bottom surface 1108 increases. Accordingly, the body 1101 can have increased lateral and/or longitudinal stability and have increased stability in footwear when compared to an inner shell without the plurality of interconnected cells 1102. Additionally, the body 1101 can flex, expand, and/or compress to adapt to the geometry and/or lacing of various footwear types. Further, the body 1101 can compress similar to a human foot during footwear donning and/or doffing so a user can don and/or doff shoes similar to donning and/or doffing shoes with a human foot.

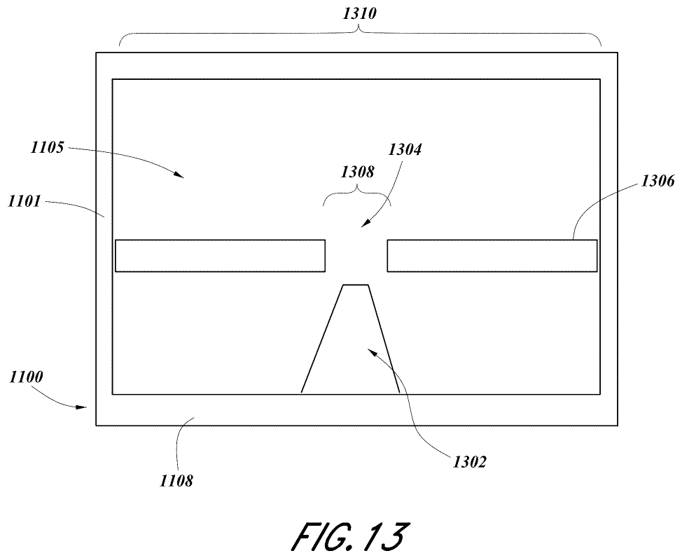

In some aspects, as shown in FIG. 13 the inner shell 1100 can include a wedge 1302 or other raised portion. The wedge 1302 can be positioned in the foot cavity 1105. The wedge 1302 can be positioned so the wedge 1302 extends into a split or slit 1304 in a prosthetic foot 1306 inserted into the foot cavity 1105. In some aspects, when the user takes a step and the bottom surface 1108 contact a walking surface, the wedge 1302 can extend further through the split or slit 1304, and the wedge 1302 can increase a width 1308 of the split or slit 1304 and a width 1310 of the prosthetic foot 1306. Accordingly, when the user takes a step, the width 1310 of the prosthetic foot 1306 can increase when the width 1108A of the bottom surface 1108 increases.

As shown in FIG. 11C, a heel portion 1110 (or at least a portion thereof) of the body 1101 of an example 3D printed inner shell 1100 can include a solid structure (i.e., not an open cell structure) so the heel portion 1110 is stiffer (i.e., more rigid) than the remainder of the body 1101 with the plurality of interconnected cells 1102 (i.e., an open cell structure). When a heel portion 1109 of the bottom surface 1108 of the body 1101 contacts a walking surface, a force may be applied to the heel portion 1109. The higher stiffness of the heel portion 1110 can provide support to the body 1101 to prevent or inhibit the body 1101 and/or the heel portion 1110 from collapsing.

During use, the inner shell 1100 may be inserted or positioned in a foot cover 102, 502. When the body 1101 flexes, expands, and/or compresses, the foot cover 102, 502 can move or translate relative to the inner shell 1100. As shown in FIG. 11D, a body 1101D of an inner shell 1100D with a heel portion 1109D that includes a plurality of interconnected cells 1102D (i.e., an open cell structure) can flex, expand, and/or compress, which can cause wear of the foot cover 102, 502. Accordingly, the solid structure of the heel portion 1109, as shown in FIG. 11C, can reduce or minimize wear of the foot cover 102, 502. With continued reference to FIG. 11C, the heel portion 1109 can include a plurality of protrusions 1112. The plurality of protrusions 1112 can include a rounded or spherical shape so the plurality of protrusions 1112 can reduce or minimize friction and/or drag between the body 1101 and the foot cover 102, 502.

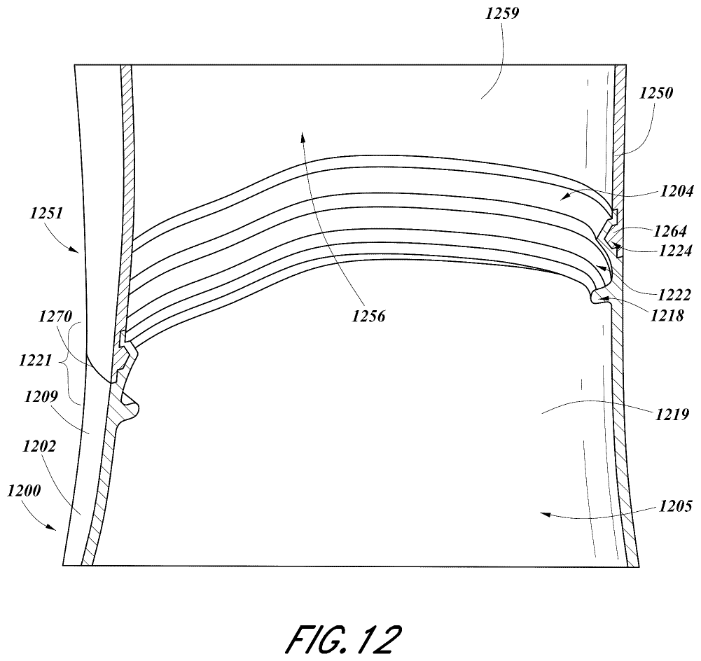

FIG. 12 illustrates an example foot cover assembly 1200 and an example leg cosmesis 1250. The foot cover assembly 100, 500 in FIGS. 1A-4C and 5A-7 can have any features of the foot cover assembly 1200, and the foot cover assembly 1200 can have any of the features of the foot cover assembly 100, 500 except for the differences described below. The leg cosmesis 550 in FIGS. 8A-9 can have any features of the leg cosmesis 1250, and the leg cosmesis 1250 can have any of the features of the leg cosmesis 550 except for the differences described below. As shown in FIG. 12, the foot cover assembly 1200 can include a foot cover 1202. The foot cover 1202 can include a channel 1222 and a recess 1224. The channel 1222 can be located on an inner surface 1219 of the foot cover 1202 a distance 1221 (for example, within 10 mm, or 8 mm, or 5 mm, or 3 mm, or otherwise) from an opening 1204 of an inner cavity 1205 of the foot cover 1202. The foot cover 1202 can include a lip 1218 (e.g., protrusion that protrudes from the inner surface 1219). The lip 1218 can form the channel 1222. The recess 1224 can be located on an outer surface 1209 of the foot cover 1202. The recess 1224 can border the opening 1204. The recess 1224 can extend into the opening 1204. Accordingly, the inner surface 1219 of the cover 1202 can extend radially into the opening 1204. The channel 1222 can secure the seal 200 in place and the recess 1224 can secure the leg cosmesis 1250 to the cover 1202. In some aspects, the channel 1222 can include one or more side rips or splits, and the channel 1222 can prevent or inhibit the lip 1218 from moving relative to the seal, which can be the seal 200 disclosed herein. For example, the foot cover 1202 may move relative to the seal throughout a gait cycle of a user as movement of the foot cover 1202 associated with walking may cause the foot cover 1202 to be pulled away from the seal. For example, the foot cover 1202 can be pulled outward when pulled downward during walking. With the one or more side rips or splits, the channel 1222 may allow the lip 1218 to flex when the foot cover 1202 moves relative to the seal 200 during the gait cycle of the user so the lip 1218 and the seal 200 maintain a pressure seal.

The leg cosmesis 1250 can include a cover 1251. The cover 1251 can include a lip 1264 (e.g., protrusion that protrudes from an inner surface 1259) located on the inner surface 1259 at or substantially near a first opening 1256 of the cover 1251. The lip 1264 can be substantially a same size and shape as the recess 1224. Accordingly, the lip 1264 can be inserted over the opening 1204 of the cover 1202 and the lip 1264 can be inserted into the recess 1224 in order to couple or secure the cover 1251 (i.e., the leg cosmesis 1250) to the cover 1202 (i.e., the foot cover assembly 1200). The cover 1251 can be press fit over the cover 1202. In some aspects, a portion of the cover 1202 located at recess 1224 can include a stiffness and the lip 1264 can include a stiffness. The stiffness of the portion of the foot cover 1202 located at the recess 1224 can be greater than a stiffness of a remainder of the foot cover 1202. The stiffness of the lip 1264 can be greater than a stiffness of a remainder of the cover 1251. In some aspects, the portion of the foot cover 1202 located at the recess 1224 can be rigid and the lip 1264 can be rigid. Accordingly, the recess 1224, the lip 1264, the opening 1204, and/or the first opening 1256 can remain substantially the same size and shape if the foot cover 1202 and/or the cover 1251 are stretched or compressed. The foot cover 1202 and the cover 1251 can create a seal 1270. The seal 1270 can include a debris, gas and/or water-tight seal. In some aspects, adhesive can be applied to the foot cover 1202 and/or the cover 1251 so the seal 1270 includes a debris, gas and/or water-tight seal. The adhesive can be applied to the recess 1224, the lip 1264, and/or the seal 1270.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise. Additionally, as used herein, “gradually” has its ordinary meaning (e.g., differs from a non-continuous, such as a step-like, change).

The scope of the present disclosure is not intended to be limited by the specific disclosures of preferred embodiments in this section or elsewhere in this specification and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A prosthetic foot cover assembly comprising:
a foot cover comprising a bottom surface, a top opening and an inner cavity, wherein the inner cavity is configured to receive a prosthetic foot through the top opening; and
a seal removably coupleable to the foot cover proximate the top opening,
wherein the bottom surface comprises a pattern of indentations, and the top opening comprises at least one protrusion, and wherein the seal has substantially a same shape as the top opening such that the seal and the at least one protrusion inhibit liquids, solids and/or gases from entering the inner cavity.

2. The prosthetic foot cover assembly of claim 1, wherein the pattern of indentations extends along an entirety of the bottom surface.

3. (canceled)

4. The prosthetic foot cover assembly of claim 1, wherein the at least one protrusion is on an inner surface of the foot cover and the at least one protrusion defines a channel that couples to at least a portion of the seal.

5. The prosthetic foot cover assembly of claim 1, wherein a leg cosmesis is coupled to the foot cover.

6. (canceled)

7. (canceled)

8. (canceled)

9. (canceled)

10. (canceled)

11. (canceled)

12. (canceled)

13. (canceled)

14. (canceled)

15. (canceled)

16. The prosthetic foot cover assembly of claim 1, further comprising an inner shell configured to receive the prosthetic foot, wherein the inner shell is disposed in the inner cavity.

17. (canceled)

18. The prosthetic foot cover assembly of claim 16, wherein the inner shell comprises a recess positioned at a heel end of the inner shell, the recess configured to receive an insert.

19. The prosthetic foot cover assembly of claim 18, wherein the inner shell comprises a first material and the insert comprises a second material, wherein the second material is stiffer than the first material.

20. (canceled)

21. The prosthetic foot cover assembly of claim 16, wherein the inner shell comprises an open cell structure.

22. (canceled)

23. (canceled)

24. (canceled)

25. (canceled)

26. (canceled)

27. The prosthetic foot cover assembly of any preceding claim 1, wherein the seal comprises a first one-way valve configured to let air into the inner cavity.

28. The prosthetic foot cover assembly of claim 27, further comprising a tube coupleable to the first one-way valve and configured to extend from the first one-way valve into the inner cavity.

29. The prosthetic foot cover assembly of claim 27, wherein the seal comprises a second one-way valve configured to let air out of the inner cavity.

30. The prosthetic foot cover assembly of claim 29, wherein the first one-way valve and the second one-way valves are configured to pressurize the inner cavity.

31. The prosthetic foot cover assembly of claim 1, further comprising one or more portions of inelastic material coupled to one or more portions of an inner surface of the inner cavity.

32. The prosthetic foot cover assembly of claim 29, wherein the first one-way valve and the second one-way valves are configured to maintain the inner cavity under a vacuum.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. An inner shell for a prosthetic foot cover assembly, the inner shell comprising:
a body comprising a plurality of interconnected cells; and
a foot cavity in the body, the foot cavity configured to receive a prosthetic foot,
wherein the inner shell is configured to be inserted into an inner cavity of a prosthetic foot cover, and wherein a cell density of the plurality of interconnected cells varies throughout the body so a stiffness of the body varies throughout the body.

45. The inner shell of claim 44, wherein each cell of the plurality of interconnected cells comprises struts, wherein the struts define an opening of each cell.

46. The inner shell of claim 44, wherein the stiffness of the body varies throughout the body to approximate a stiffness a human foot.

47. The inner shell of any of claims 44, wherein the body comprises a bottom surface, the bottom surface comprising a width and a length, and wherein each cell of the plurality of interconnected cells flexes, expands, and/or compresses so the width and/or the length increase when a force is applied to the bottom surface.

48. (canceled)

49. The inner shell of claim 44, wherein a heel portion of the body comprises a plurality of protrusions.

50. (canceled)

51. The inner shell of any of claims 44, wherein the cell density of the plurality of interconnected cells is determined based on a stiffness and/or an anatomy of a foot of a user.

52. (canceled)
